(19) United States
(12) Patent Application Publication
(10) Pub. No.: US 2020/0188228 A1
(43) Pub. Date: Jun. 18, 2020

(54) Equipment and Method for Buoyancy Assisted Psychotherapy

(71) Applicant: Wesley R. Easley, Petaluma, CA (US)

(72) Inventor: Wesley R. Easley, Petaluma, CA (US)

(21) Appl. No.: 16/224,307

(22) Filed: Dec. 18, 2018

(51) Int. Cl. A61H 35/00; A61M 21/00; A61M 21/02

(52) U.S. Cl. CPC A61H 35/00; A61M 21/0094; A61M 21/02; A61M 2209/088; A61H 2201/10; A61M 2021/0022; A61M 2021/0027; A61H 2035/004

(57) ABSTRACT

Equipment and a process for creating an environment in which to conduct a successful psychotherapy session with a subject who has a chronic pain condition employing a water tank and a device for inducing alternating tactile or audible stimulation.

### CROSS-REFERENCES TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### 1. Field of the Invention

The present invention relates generally to a method to aid the psychotherapy process for subjects with chronic pain conditions. More specifically, the present invention relates to a process for conducting psychotherapy while the subject is submerged in an aquatic environment.

### 2. Description of the Related Art

A common treatment process for subjects with Post-traumatic Stress Disorder (PTSD) is Eye Movement Desensitization and Reprocessing (EMDR) in which the therapist instructs the subject to recall a traumatic event while rapidly moving their eyes back and forth. Although generally a successful method, there are inherent disadvantages with this process such as maintaining consistent eye movement over time or interference in the process due to crying. To overcome these disadvantages, alternate methods of creating stimuli have been developed, including using tones in alternating ears or providing a tactile stimulation to different sides of the body, which might be done by the therapist with gentle taps to the hands.

Unfortunately, many subjects who suffer from PTSD also have physical injuries that result in chronic pain conditions. Multiple studies have shown that when a person is in physical pain, their ability to focus and concentrate becomes significantly impaired. In the realm of psychotherapy, therapists can have great difficulty helping their subjects in this condition because the subjects are unable to concentrate and contribute to the session. Traditional methods for conducting psychotherapy sessions generally include having the subject sit or lie down in a quiet and comfortable environment while discussing their issues. When the subject is in pain in this standard type of setting, the process is compromised and the session is less likely to be successful.

The present invention resolves this issue by providing a process that assists the subject's ability to think and concentrate during a psychotherapy session while allowing the therapist to employ variations of the EMDR procedure. The process presented is unique in that it employs an aquatic environment to help relieve the physical pain. When the subject is submerged in water or a saline solution, the hydrostatic pressure decreases pain and edema, reducing the interruptive effect of pain signals to cognitive processing. Of course, when a subject is submerged in water, a traditional therapy session becomes more difficult. For example, the therapist would not be able to assist with eye movement or touch the client on alternating hands. The present invention resolves these issues by providing the appropriate equipment to allow variations of the EMDR procedure to be conducted while the subject is submerged.

With the pain condition reduced or eliminated, the subject is able to concentrate and focus on the psychotherapy session, and with the addition of the alternating stimuli, the subject is able to benefit from the EMDR process. Initially, this process was developed to address the needs of individuals with spinal injuries, such as spinal fractures, nerve damage, degenerative disc disease, and disc herniation, but it can be employed for subjects who experience pain for any other reason where the discomfort is alleviated by an aquatic environment.

### BRIEF SUMMARY OF THE INVENTION

The present invention comprises equipment and a process for providing an environment in which to conduct a successful psychotherapy session with a subject who experiences a chronic pain condition. The equipment includes a water tank large enough for a single person to sit or stand in while submerged to the chest or neck in water or a saline solution and a waterproof device that creates alternating stimuli to engage both the right and left side of the subject's brain. The stimulus device consists of a waterproof transmitter, two gloves that each contain one waterproof receiver in the palm of the glove that vibrates when activated, and earphones that can receive audio signals from the transmitter. The transmitter is adjustable such that the psychotherapist can control the duration, timing and intensity of vibration in the receivers, and tones and volume in the earphones.

The process begins with the subject entering the water tank and filling the tank with body temperature water or saline solution to the height of the subject's chest or neck. This aquatic environment alleviates the subject's physical pain by reducing the gravitational impact on the body. The subject then dons the gloves or headphones and the transmitter is turned on and sends alternating signals to the receivers or the headphones. The alternating stimuli engage both the right and left side of the subject's brain which aids in therapy by allowing the subject to fully recall a past traumatic event. If the therapist will use sound coupled with the alternating tactile stimuli, for example to trigger the subject's sensitivity to an issue, the transmitter can also be programmed to play the stimulus sound. The therapist can then proceed with the therapeutic session.

### BRIEF DESCRIPTION OF THE SEVERAL VIEWS OF THE DRAWINGS

A more complete understanding of the present invention may be derived by referring to the detailed description and claims when considered in connection with the figures, wherein like reference numbers refer to similar items throughout the figures and:

FIG. 1 is flow chart of the process employing tactile stimuli;

FIG. 2 is a view of the transmitter;

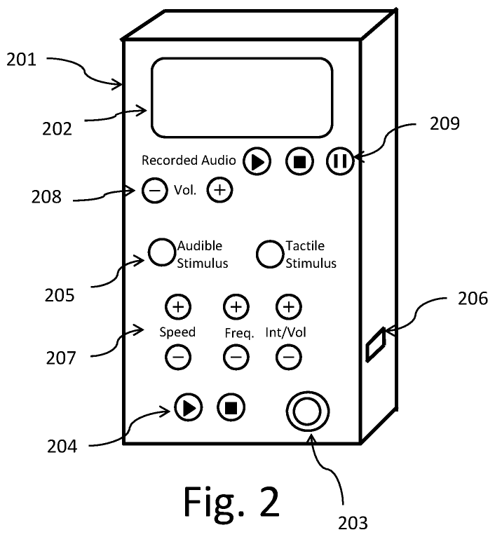

FIG. 3 is a view of the gloves showing the location of the tactile stimulators;

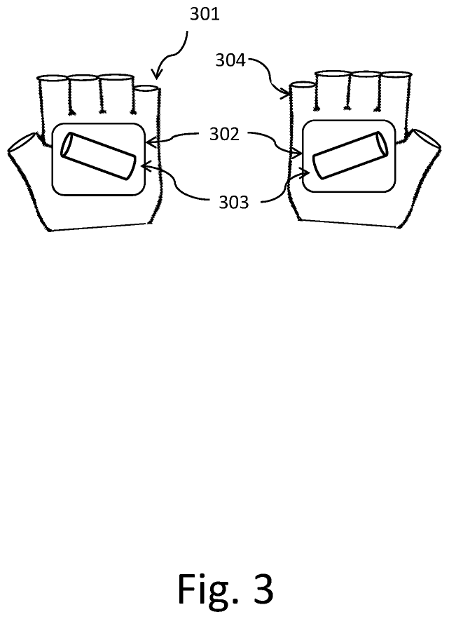

FIG. 4 is a view of a subject standing in the water tank;

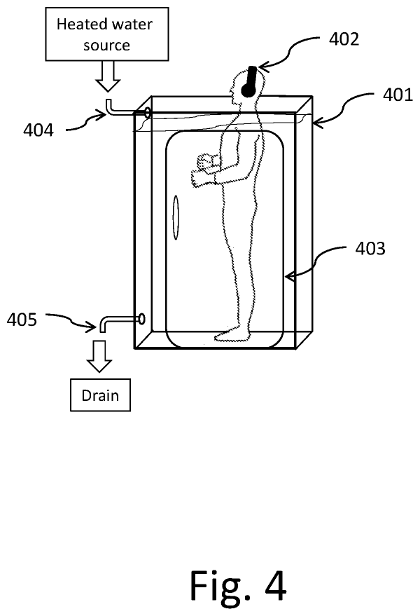

FIG. 5 is a view of a subject seated in a water tank;

FIG. 6 is a cross-section of the vibration device; and

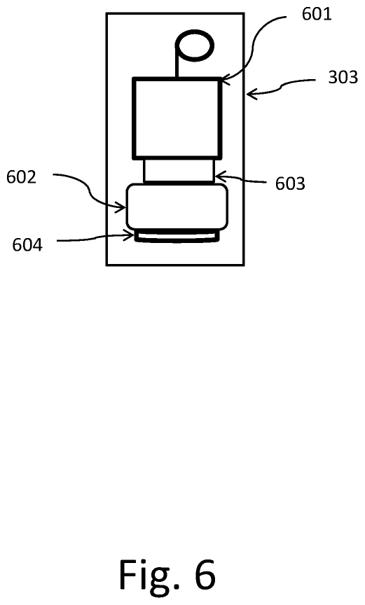

FIG. 7 is a flow chart of the process employing audible stimuli.

### DETAILED DESCRIPTION OF THE INVENTION

In the following detailed description of the invention, reference is made to the accompanying drawings which form a part of the disclosure and, which show by way of illustration, and not of limitation, specific embodiments by which the invention may be practiced. The drawings, the foregoing discussion, and the following description are exemplary and explanatory only, and are not intended to limit the scope of the invention or its application in any manner.

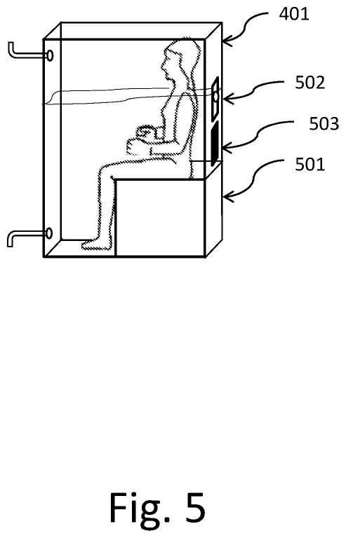

The present invention comprises equipment and a process for providing an environment in which to conduct a successful psychotherapy session with a subject who experiences a chronic pain condition. The equipment consists of a Water Tank 401, a Transmitter 201, a right Glove 301, a left Glove 304, two Palm Stimulators 303, and a set of commercially available waterproof Headphones 402. The Water Tank 401 is large enough for a single person to stand in and is constructed of appropriate strength material to be filled with water such that the subject is submerged to the neck. The Water Tank 401 preferably has a watertight Door 403 for ease of entry, an Inlet 404 for water from a heated water source and a Drain 405 so the tank can be emptied. In an alternate configuration, the Water Tank 401 may have a Seat 501 so the subject may remain seated during the session. In yet another alternate configuration, as shown in FIG. 5, the Water Tank 401 does not have a door for entry and instead is accessed from the top. In this configuration, the Water Tank 401, would have Controls 502 and a Heating Element 503, so the water temperature could be adjusted to body temperature prior to the subject entering the Water Tank 401.

The Right Glove 301 and Left Glove 304 are made of waterproof material such as neoprene and each has a Pocket 302 sewn into its palm that is large enough to hold a Palm Stimulator 303. The Palm Stimulator 303 is a waterproof cylinder containing a Vibration Device 601 connected to a Receiver 602 via a Circuit Board 603. The assembly is powered by a Battery 604. The Vibration Device 601 is preferably a motor with an offset weight, and the Circuit Board 603 contains the appropriate commercially available electronics to convert signals received by the Receivers 602 to output as short bursts of vibration in the Vibration Device 601. The Receivers 602 are preferably Bluetooth devices, but any other wireless technology may be employed. The Receivers 602 are adjusted to different frequencies so they can be operated independently by the same Transmitter 201.

The Transmitter 201 is a wireless handheld device with a USB Port 206 for providing pre-recorded audio input. It is preferably a Bluetooth device, but any other wireless technology may be employed. It is battery powered and has an On/Off Power Button 203. It has Selection Buttons 205 which are used by the therapist to select either an audible stimulus or a tactile stimulus, and Up/Down Selectors 207 for adjustment of speed, frequency, and intensity or volume for the stimuli. It has Play and Stop Buttons 204 for the stimuli. It also has Volume Control Buttons 208 and Play, Stop and Pause Buttons 209 for recorded audio that can be input via a thumb drive at the USB Port 206. For the recorded audio, the play button re-starts the audio from the beginning and the pause button allows a pause and restart from the same point in the recording. The Transmitter 201 has a display screen 202 which shows control levels, settings and status. The entire device is preferably waterproof, or, in the alternative, has a waterproof case that is made of thin plastic such that the buttons can be operated through it, and that is configured to accommodate a thumb drive installed in the USB Port 206. The Transmitter 201 uses standard internal electronic components to transmit signals independently to each Receiver 602 and the Headphones 402.

The Headphones 402 are commercially available waterproof wireless headphones that will receive the audio signals from the Transmitter 201. In an alternate configuration, the Headphones may be replaced by waterproof wireless earbuds.

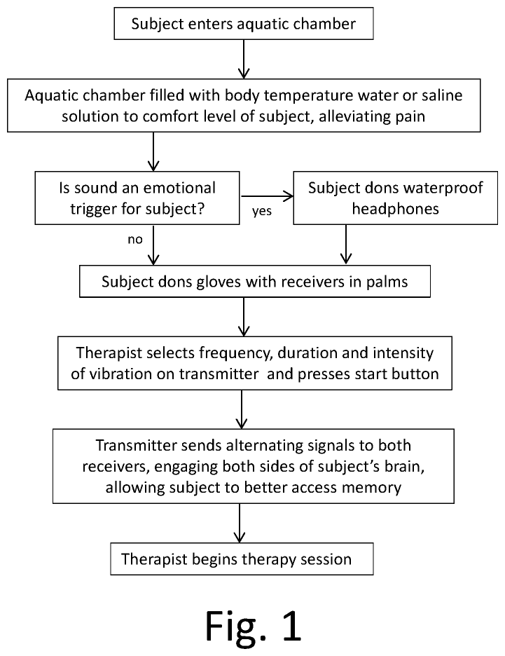

As shown in the flow chart in FIG. 1, the process begins with the subject entering the Water Tank 401. The Water Tank 401 is filled with body temperature water or saline solution to the height of the subject's chest or neck so that the subject's physical pain is reduced. The subject then dons the Gloves 301 and 304. The therapist turns on the Transmitter 201 and selects Tactile Stimulus using the Selection Buttons 205. The therapist adjusts the speed, frequency, and intensity of the tactile stimuli to settings appropriate for the subject using the Up/Down Selectors 207. The therapist then hits the play button to start the transmission. This causes the Transmitter 201 to send alternating signals to the Receivers 602 in accordance with the settings established by the therapist. The alternating signals cause the Palm Stimulators 303 to vibrate in alternating bursts. This alternating stimuli in the subject's palms engages both the right and left side of the subject's brain, allowing the subject to fully recall a past traumatic event. The therapist may then begin the therapy session.

If the therapist will use sound in the therapy session, for example to trigger the subject's sensitivity to a noise, the therapist inserts a thumb drive with the appropriate sound into the Transmitter 201, and the subject dons the waterproof Headphones 402. The therapist then uses the Play, Stop and Pause controls 209 to play the stimulus sound.

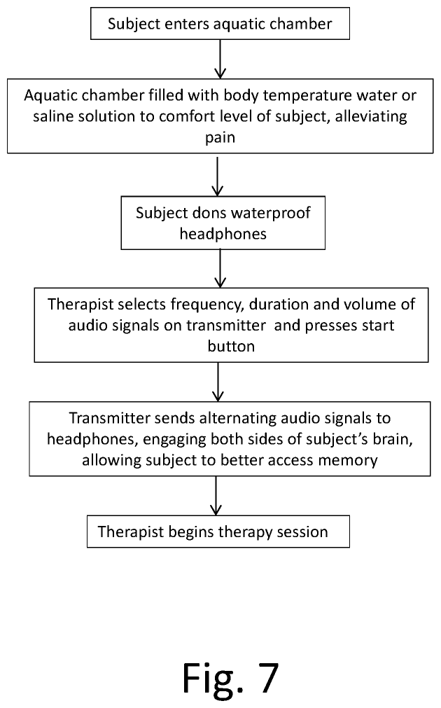

In an alternate process, as shown in the flow chart in FIG. 7, the therapist uses alternating audible stimuli rather than tactile stimuli to engage both sides of the subject's brain. In this case, the Gloves 301 and 304 are not required, and the subject merely dons the Headphones 402 after entering the Water Tank 401. The therapist selects Audible Stimulus using the Selection Buttons 205 and adjusts the alternating stimuli which will be in the form of tones to the Headphones 402 using the Up/Down Selectors 207.

Many modifications and variations of this process and equipment may be made without departing from its spirit and scope, as will be appreciated by those skilled in the art. The specific embodiments described herein are offered by way of example only. The embodiments were chosen and described in order to best explain the principles of the invention and its practiced applications.

What is claimed is:

1. Equipment for providing an environment in which to conduct a successful psychotherapy session with a subject with a chronic pain condition comprising:
a waterproof right glove containing a right side wireless vibration device in the palm;
a waterproof left glove containing a left side wireless vibration device in the palm which receives signals on a different frequency than the right side wireless vibration device;
a water tank filled with body temperature water that is large enough for the subject to enter and be submerged to the neck;
and a controller capable of sending wireless signals that cause the right side wireless vibration device and the left side wireless vibration device to vibrate in an alternating pattern.

2. The equipment of claim 1 with a set of wireless waterproof headphones and a controller capable of sending pre-recorded sounds to the headphones that are useful for the psychotherapy session.

3. Equipment for providing an environment in which to conduct a successful psychotherapy session for a subject with a chronic pain condition comprising:
a set of wireless waterproof headphones with a right side ear piece and a left side ear piece;
a water tank filled with body temperature water that is large enough for the subject to enter and be submerged to the neck;
and a controller capable of sending wireless signals to the headphones creating tones in the rights side ear piece and left side earpiece in an alternating pattern.

4. A method for a therapist to conduct a psychotherapy session with a subject with a chronic pain condition comprising the steps of:
the subject donning a waterproof right glove containing a right side wireless vibration device in the palm;
the subject donning a waterproof left glove containing a left side wireless vibration device in the palm which receives signals on a different frequency than the right side wireless vibration device;
the subject entering a water tank filled with body temperature water that is large enough for the subject to enter and be submerged to the neck;
filling the water tank with body temperature water to a level at which the buoyancy of the water lessens the subject's chronic pain condition;
using a controller capable of sending wireless signals that cause the right side wireless vibration device and the left side wireless vibration device to vibrate in an alternating pattern, the therapist sending alternating wireless signals to cause the right side wireless vibration device and the left side wireless vibration device to vibrate in an alternating pattern; and
conducting the psychotherapy session.

5. A method for a therapist to conduct a psychotherapy session with a subject with a chronic pain condition comprising the steps of:
the subject donning waterproof wireless headphones;
the subject donning a waterproof right glove containing a right side wireless vibration device in the palm;
the subject donning a waterproof left glove containing a left side wireless vibration device in the palm which receives signals on a different frequency than the right side wireless vibration device;
the subject entering a water tank filled with body temperature water that is large enough for the subject to enter and be submerged to the neck;
filling the water tank with body temperature water to a level at which the buoyancy of the water lessens the subject's chronic pain condition;
using a controller capable of sending wireless signals that cause the right side wireless vibration device and the left side wireless vibration device to vibrate in an alternating pattern, the therapist sending alternating wireless signals to cause the right side wireless vibration device and the left side wireless vibration device to vibrate in an alternating pattern;
using a controller capable of sending wireless signals to the headphones, the therapist sending sounds that trigger the subject's memories; and
conducting the psychotherapy session.

6. A method for a therapist to conduct a psychotherapy session with a subject with a chronic pain condition comprising the steps of:
the subject donning waterproof wireless headphones with a left side earpiece and a right side earpiece;
the subject entering a water tank filled with body temperature water that is large enough for the subject to enter and be submerged to the neck;
filling the water tank with body temperature water to a level at which the buoyancy of the water lessens the subject's chronic pain condition;
using a controller capable of sending wireless signals to the right side earpiece of the headphones and the left side earpiece of the headphones in an alternating pattern, the therapist sending alternating wireless signals to cause tones to sound in the right side earpiece and left side earpiece in an alternating pattern; and
conducting the psychotherapy session.
